(19) United States
(12) Patent Application Publication
(10) Pub. No.: US 2009/0326488 A1
(43) Pub. Date: Dec. 31, 2009

(54) Canister, Suction Device and System For Vacuum Treatment Securing a Fixed Treatment Pressure

(76) Inventors: Klaus Budig, Olstykke (DK); Nicolai Sorensen, Hellebaek (DK)

Correspondence Address: JACOBSON HOLMAN PLLC, 400 SEVENTH STREET N.W., SUITE 600, WASHINGTON, DC, 20004, US

(21) Appl. No.: 12/309,596

(22) PCT Filed: Jul. 20, 2007
(86) PCT No.: PCT/EP2007/057535

§ 371 Date: Aug. 21, 2009

(30) Foreign Application Priority Data
Jul. 24, 2006 (DK) PA 2006 01011

(51) Int. Cl. A61M 1/00

(52) U.S. Cl. 604/319

(57) ABSTRACT

A canister, suction device and system for collection of effluents, in particular exudates, from a wound during vacuum treatment comprises a pump head (112) and a regulator (120) regulating the pressure in the wound (102) to secure a fixed treatment pressure. The regulator (120) may regulate the pressure by sensing the pressure in the wound and in response thereto control operation of the pump head. The regulator (120) may be permanently integrated in a housing of the canister (114) in a non-removable and/or non-accessible manner to exclude the possibility of setting adjusting the fixed treatment pressure. Air may be allowed into the wound if the pressure at the wound (102) is determined to be less than a threshold pressure.

### TECHNICAL FIELD

The present invention relates to a system for vacuum treatment, a suction device for removal of effluents, in particular exudates, and a canister for collection of effluents, in particular exudates from a wound.

### BACKGROUND OF THE INVENTION

The prior art contemplates that open wounds may be treated by providing vacuum in the space above the wound to promote healing. This process is often described as Vacuum Treatment or Suction Wound Drainage.

Vacuum treatment is a complex treatment to be carried out e.g. by a nurse in a hospital due to the many steps to be carried out before the actual vacuum treatment can be initiated. The nurse has to make sure that the wound is sealed tightly with a draining device, such as a suction head, that allows for fluids from the wound to be sucked away from the wound. The nurse also has to see to that the draining device is properly connected with a suction device. The draining device may be connected directly with the suction device or the draining device may be connected via a conduit to the suction device. Further more, the nurse has to make sure that the rather complex suction device provides the right treatment pressure, prescribed by the doctor, since this is crucial for the effect of the treatment.

A precise control of the pressure level in vacuum treatment systems is desired to ensure appropriate wound treatment. However, pressure control is often complicated by leakages in the wound enclosure or in conduits connecting the wound with the pump, or by occlusions caused solid or semi-solid matter drawn from the wound. Removal of an occlusion may take unacceptably long, and sometimes even requires interruption of the pump operation to exchange or rinse a conduit, if the pump is not capable of generating a sufficiently high pressure drop across the occlusion to overcome mechanical forces keeping the occlusion in place. It has been found particularly difficult to remove occlusions at far distal portions of a conduit connecting the wound with a source of suction, i.e. portions of a conduit which are immediately next to the wound or even within the wound.

It is an object of preferred embodiments of the invention to provide a vacuum treatment system, which is easy to install. It is a further object of preferred embodiments of the invention to provide a vacuum treatment system, which allows the wound pressure to be precisely and rapidly controlled in an automatic manner, in particular in the case of occlusions at distal portions of connecting conduits.

### SUMMARY OF THE INVENTION

In a first aspect of the invention a canister for collection of effluents, in particular exudates, from a wound during vacuum treatment is provided, said canister comprising, an outlet to the surroundings, a wound inlet, a pump head having a pump inlet and a pump outlet, and a regulator regulating the pressure at the wound, the regulator securing a fixed treatment pressure. In this way a canister is provided that ensures that the treatment pressure cannot be changed after the set up of a system for vacuum treatment comprising the canister. This is beneficial since this reduces the risk of mistreatment because it is not possible for e.g. a patient or another person to change the treatment pressure after the set up. In one embodiment the regulator is permanently secured to or integrated in the canister. Preferably, it is non-detachably integrated within a housing of the canister. In the present context, the term “fixed treatment pressure” may imply that there the possibility of setting or adjusting the treatment pressure is excluded, once the canister has been manufactured. Hence a fixed treatment pressure may be understood as a non-modifiable treatment pressure.

The pump head typically comprises mainly mechanical parts and will in use be connected to a drive unit. It is in the pump head that a negative pressure or even vacuum is created. The drive unit operates the pump head, so that a flow is created in the pump head. The flow rate created by the pump head is regulated by the drive unit.

A fluid from a wound may comprise effluents and exudates.

In an embodiment of the canister, the regulator further comprises a regulator element and the fixed treatment pressure can be chosen among a number of treatment pressures in the regulator, and that the chosen treatment pressure is secured by the regulator element thereby securing the fixed treatment pressure to be the chosen treatment pressure. Preferably the canister only allows for one fixed treatment pressure since this reduces the number of steps the nurse has to carry out when setting up the system for vacuum treatment, consequently also reducing the risk of choosing the wrong treatment pressure. On the other hand if the regulator allows for choosing among a number of treatment pressures only one type of canister may be kept on stock.

In yet another embodiment of the invention a canister wherein the regulator regulates the pressure by sensing the pressure in the wound and in response hereto regulates the operation speed of suction of the pump head is provided.

The regulator may be arranged to allow air into the wound if the pressure at the wound is less than the fixed treatment pressure, so as to increase the pressure in the wound. Hence, an occlusion may be efficiently removed, as the occlusion is not only affected by negative pressure downstream of the occlusion, but also by a positive pressure upstream of the wound. For example, ambient air may be drawn into the wound and from there into a conduit connecting the wound with a suction source. Hence, a larger pressure difference exists across the occlusion than if pressure upstream of the occlusion was kept at the negative level it used to be before the occlusion occurred. In other words, the occlusion is not only sucked through the conduit by a downstream suction pressure, but also pushed by positive pressure at the upstream side of the occlusion.

It is envisaged that a source of positive pressure may be provided to apply a pressure above atmospheric pressure to the upstream side of the occlusion. For example, a pressure side of the pump providing suction in the system may be connected to the regulator to provide a positive pressure. Alternatively, a separate pump may be provided as a positive pressure source.

In one embodiment of a canister the regulator further comprises a regulator spring and a regulator outlet, such that if in use the pressure in the wound is less than the fixed treatment pressure, air is allowed through the regulator outlet and into the wound thereby increasing the pressure in the wound. This is an advantage since a canister like this can be connected to a very simple drive unit only allowing one operation speed of the pump head.

In still another embodiment of the invention a canister wherein the regulator further comprises a filter element thereby securing that only non-contaminated gas is let through the regulator outlet is provided. The filter element could e.g. be an odour filter, an antibacterial filter or combinations hereof. By filtering the fluid unwanted characteristics, like odour or bacteria, of the fluid can be reduced and maybe even removed.

In an embodiment a canister wherein the wound inlet and the pump inlet are connected such that the pressure in the canister is approximately the same as the pressure in the surroundings is provided. It is an advantage to use such a canister since it allows for a larger choice of material of the shell of the canister. E.g. may the shell be made of a hard material such as a hard plastic, or the shell can be made of soft material e.g. so that is constitutes a bag such as a plastic bag. Further the fluids from the wound is lead through the pump head and in this way dress the pump head thus providing for a better performance of a system for vacuum treatment comprising such a canister.

Another embodiment provides a canister wherein the wound inlet is connected to a part of the shell of the canister such that in use the pressure inside the canister is less than the pressure at the surroundings, and that the pump inlet is in contact with the inside of canister, such that in use canister fluid is sucked into the pump head via the pump inlet and wound fluid is sucked from the wound out through the wound inlet to the inside of the canister thus mixing the wound fluid into the canister fluid.

In a second aspect of the invention a suction device for removal of effluents, in particular exudates, from a wound during vacuum treatment is provided, the suction device comprising a canister with a wound inlet, a pump head having a pump inlet and a pump outlet, a drive unit operating the pump head, an outlet to the surroundings, and a regulator regulating the pressure at the wound, the regulator securing a fixed treatment pressure.

An embodiment provides a suction device, wherein the regulator further comprising a regulator element and wherein the fixed treatment pressure can be chosen among a number of treatment pressures in the regulator and that the chosen treatment pressure is secured by the regulator element thereby securing the fixed treatment pressure to be the chosen treatment pressure.

In another embodiment a suction device is provided, wherein the regulator regulates the pressure by sensing the pressure in the wound and in response hereto regulates the operation speed of suction of the pump head.

The regulator may be arranged to allow air into the wound if the pressure at the wound is less than the fixed treatment pressure, so as to increase the pressure in the wound. Hence, an occlusion may be efficiently removed, as the occlusion is not only affected by negative pressure downstream of the occlusion, but also by a positive pressure upstream of the wound. For example, ambient air may be drawn into the wound and from there into a conduit connecting the wound with a suction source. Hence, a larger pressure difference exists across the occlusion than if pressure upstream of the occlusion was kept at the negative level it used to be before the occlusion occurred. In other words, the occlusion is not only sucked through the conduit by a downstream suction pressure, but also pushed by positive pressure at the upstream side of the occlusion.

It is envisaged that a source of positive pressure may be provided to apply a pressure above atmospheric pressure to the upstream side of the occlusion. For example, a pressure side of the pump providing suction in the system may be connected to the regulator to provide a positive pressure. Alternatively, a separate pump may be provided as a positive pressure source.

In one another embodiment a suction device, wherein the regulator further comprises a regulator spring and a regulator outlet, such that if in use the pressure in the wound is less than the fixed treatment pressure, air is allowed through the regulator outlet and into the wound thereby increasing the pressure at the wound.

In still another embodiment a suction device is provided, wherein the regulator comprises a filter element thereby securing that non-contaminated gas is let through the regulator outlet.

Another embodiment provides a suction device, wherein the canister is according to the first aspect of the invention.

In a third aspect of the invention a system for vacuum treatment of a wound is provided, the system comprising a suction device according to a second aspect of the invention, a wound cover, and a conduit part connecting the wound cover and the wound inlet.

An embodiment of the system for vacuum treatment of a wound is provided, wherein the system comprises a suction device, a wound cover, and a conduit part connecting the wound cover and the wound inlet, the conduit part comprising a first conduit and a second conduit. The first conduit may e.g. be used for suction of fluid from the wound and the second conduit may be used for regulating the pressure in the wound.

In still another embodiment of the invention a system is provided, further comprising a suction head at least partly placed under the wound cover. The suction head may be connected to the conduit part.

In another embodiment of a system for vacuum treatment, the conduit part comprises a suction head at least partly placed under the wound cover, a first conduit and a second conduit, the first conduit being connected with the suction head.

The invention further provide a method for removal of fluids from a wound, the method comprising:
providing a wound cover which is attachable to a wound circumference of a living being thereby to form an enclosure thereby sealing the wound site from the ambient air,
bringing a pump in fluid communication with the enclosure via a first tube to provide a pressure difference between a suction pressure in the enclosure and an air pressure of an ambient space,
providing a first valve, which is capable of providing fluid communication between the first tube and the ambient air,
   wherein said valve is intermittently operated to provide a gas flow through the first tube.

In all aspects of the present invention, the wound cover may e.g. form a cavity with an opening surrounded by an edge portion which facilitates an essentially gas tight transition to the skin of the treated living being, e.g. via a soft resilient adhesive material. The wound cover may be occlusive or semi-occlusive, e.g. being vapor permeable but water impermeable. The pump may produce a pressure difference, which is sufficient to draw liquids away from the wound, e.g. a negative pressure in the range of 10 to 600 mm Hg. The negative pressure may be determined as a relative pressure or as an absolute pressure. To determine a relative pressure, the air pressure of ambient space is the surrounding atmospheric air pressure, and to determine an absolute pressure, the air pressure of ambient space is a reference air pressure of a reference space, i.e. an enclosed volume that has a pressure relative to which the vacuum pressure is measured.

Pressure detection means may further be included. The pressure detection means are typically located remote from the wound, e.g. in connection with a pump head, driver unit or control unit.

The pressure over the wound may be kept substantially constantly negative or it may alternate between different values of negative pressure and the pressure of the ambient air. It is preferred to control the pressure fit in a window defined by an upper and a lower level. The effluent from the wound to be removed by suction is in the form of a fluid comprising a mixture of liquid and gas. When this mixture enters the first tube connecting the pump and the wound enclosure, the liquid may from time to time fill the tube to form a liquid string and thus block the free passage of gas through the tube. This may influence on the pressure in the enclosure as well as on the measurement of the pressure, as it may give rise to fluctuations in the pressure over the wound and inaccurate determination of the pressure.

The present method surprisingly provides a solution to this problem by incorporating a first valve that can be operated to provide a connection between the first tube and the ambient air. The valve may be opened for only a short period, thus providing a gas stream through the conduit between the enclosure and the pump, establishing free passage of air and providing a reliable determination of the pressure. The valve may be open long enough to provide a gas stream, but short enough to maintain a negative pressure in the enclosure.

Preferably, the valve may be operated while the pump is working in order not to increase pressure in the enclosure.

The operation of the valve may be automatic, activating the valve within regular intervals or the valve may be operated by a signal from the pressure detection means or a control unit. The valve may be activated when a predetermined level of pressure is reached, if the upper level is reached, the valve may be opened, if the lower level is reached, the valve may be closed and optionally a signal for the pump to increase negative pressure may be sent.

Pressure sensing means may not be provided, but the system may be adjusted to a satisfactory pressure level, and intermittently activation of the valve may serve to maintain the pressure level.

The valve may be opened for a short period to purge the first tube, and a measurement of the pressure may be conducted immediately after, to achieve a correct pressure determination.

In one embodiment of the invention the system comprises a control unit. The control unit receives information about the pressure, the pump and the valve, and combines the information to adjust the activity of the pump and/or valve in order to maintain the desired pressure over the wound.

In one embodiment of the invention the first valve may be manually operated.

The first valve is preferably located at the end of the first tube being connected to the enclosure. This location enables a gas flow through substantially the entire first tube when the first valve is activated, thus purging the tube from any liquid strings.

Contrary to systems where a bleeding hole is established on the pump-enclosure conduit, the first valve of the present may be operated to be in a closed or open position. Thus, the pump may be stopped when sufficient negative pressure is achieved, and started again when the negative pressure decreases. A bleeding hole is constantly open, and requires constant work of the pump in order to maintain negative pressure. Constant operation of the pump is power consuming and may be noisy and can be a nuisance to the patient.

The first valve may be connected to the first tube via a second tube. The tube attached to the wound enclosure may be divided into a fork of a first tube, connecting to the pump, and a second tube connecting to the first valve. Thus, the valve is not in direct contact with the first tube and the fluids passing through this, and the risk of clotting the valve may be reduced, as well as the valve may be closer to or integrated in the rest of the system.

In order to avoid a decrease in the negative pressure in the enclosure when the first valve is opened, the first tube may be provided with a second valve between the enclosure and the first valve. This second valve is preferably closed when the first valve is open. Such second valve may not be necessary if the pump is strong enough, as this may maintain the pressure over the wound even when the first valve is open.

The second valve may be operated manually or by a signal from the first valve or the pressure detection means or control unit, but preferably the valve is opened/closed automatically by the pressure in the tube. In a preferred embodiment of the invention the second valve is open when the pressure in the enclosure is higher than the pressure in the tube. A suitable valve for such purpose may be a one-way valve, e.g. a duck valve.

The method may further comprise providing a canister for receiving the liquid, and separating the gas from the liquid of the wound fluid. The canister is preferably located in connection with the pump. The pump may comprise a pump head for leading the fluid and a drive unit for driving the pump. The canister may be located before or after the pump. The canister may comprise absorbent material.

The method may further comprise providing separating means, for separating the fluid into gas and liquid.

The pressure detection means may produce a signal to the control unit or to the first valve when a predetermined pressure level is reached.

The invention further provides to a suction system for removal of fluids from a wound site, the system comprising:
a wound cover, which is attachable to a wound circumference of a living being thereby to form an enclosure thereby sealing the wound site from the ambient air,
a pump in fluid communication with the enclosure to provide a pressure difference between a suction pressure in the enclosure and an air pressure of an ambient space,
a first tube connecting the enclosure to the pump, and
a first valve being capable of providing fluid communication between the first tube and the ambient air.

The system may be provided with pressure detection means.

The system may further comprise a control unit, controlling one or more of the operations of the system, e.g. recording the pressure and adjusting the activity of the pump and/or the valve in order to maintain a predetermined negative pressure.

The first valve may be connected to the first tube via a second tube.

The first tube may comprise a second valve located between the enclosure and the first valve.

The second valve may be closed when the first valve is open or the second valve may be open when the pressure in the enclosure is higher than the pressure in the tube.

A canister may be provided in the system, the canister may e.g. be located before or after the pump.

The invention will now be further described with reference to the accompanying drawings, in which:

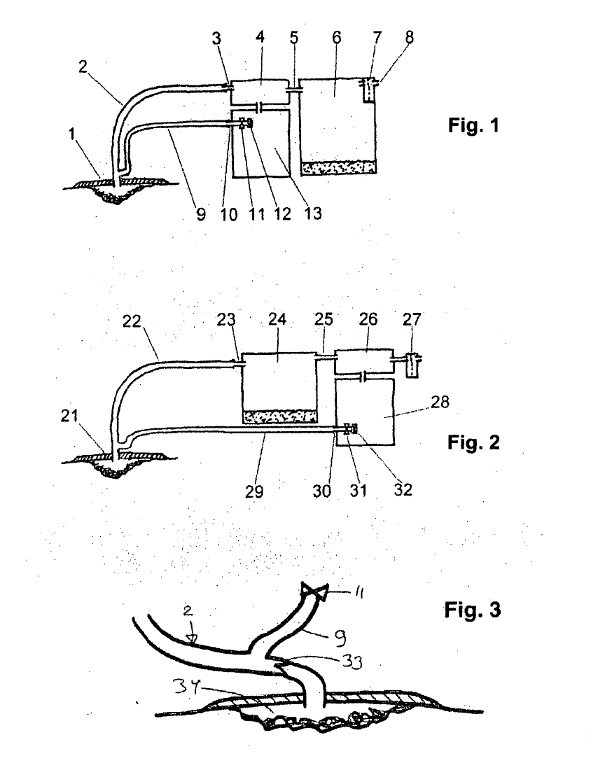

FIG. 1 shows one embodiment of the invention,

FIG. 2 shows another embodiment of the invention,

FIG. 3 shows a detailed view of a part of the invention,

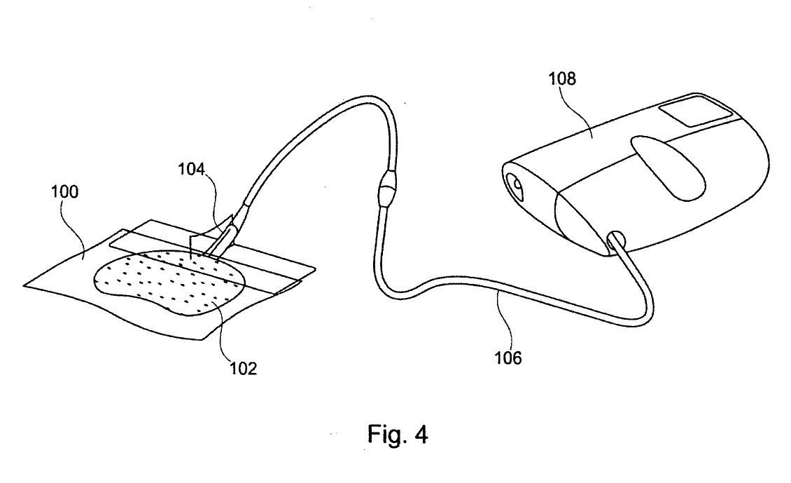

FIG. 4 is illustrates an embodiment of the invention,

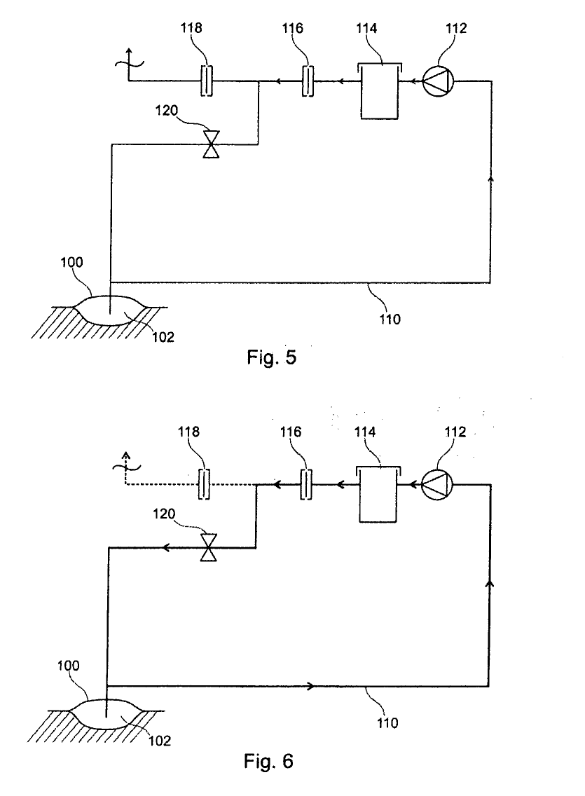

FIGS. 5-11 are operational charts of embodiments of the invention,

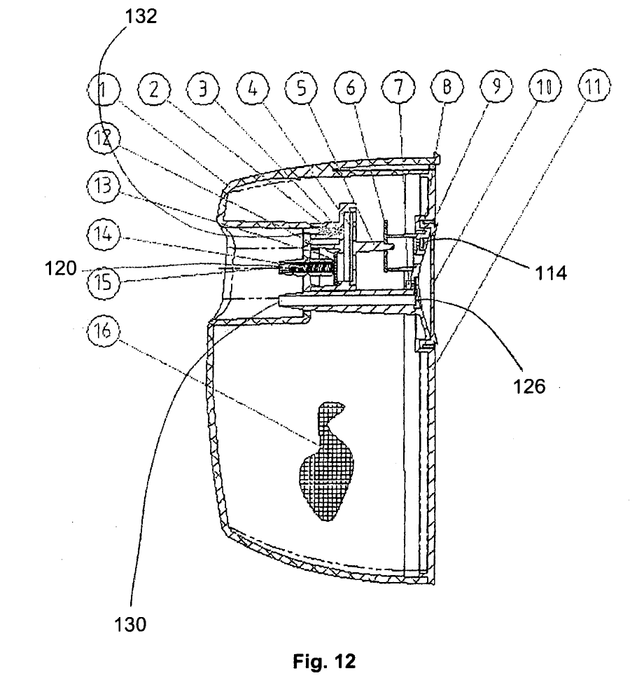

FIG. 12 illustrates an embodiment of a canister according to the invention.

FIG. 1 shows a schematic drawing of the system. The wound cover (1) is sealing the wound to form an enclosure. Fluid in the form of air and liquid wound exudate are transported by suction through a first tube (2) by negative pressure created by the pump head (4) and drive unit (13). The first tube (2) may comprise several parts and different fittings can be added.

The first tube (2) is connected to the pump head (4) by an inlet (3). The air and exudate are transported through the pump head (4) and by an outlet (5) to a canister (6). The canister can be any kind of volume suitable for collecting and/or separating air and liquid, such as a flexible bag.

In the canister (6), air and liquid are separated into two phases. The air will pass through the outlet (8) and the liquid will be retained in the canister (6). The canister may contain an absorbent component.

The outlet (8) of the canister may be provided with a filter (7) for removal of undesired components of the effluent gas.

The pump head (4) is connected to the drive unit (13). The pump head may be disposable.

A second tube (9) is connected to the first tube near the enclosure in one end and by an inlet (10) to a first valve (11) and filter (12) in the other end.

The first valve (11) will open and air will pass through the filter (12), second tube (9), first tube (2) and further through the rest of the system thus enabling free passage of gas through the first tube (2) and removal of any liquid strings.

Liquid strings in tubes/drains are contributing to an inaccuracy of the negative pressure in the system by inducing fluctuations in the pressure over the wound. These fluctuations are minimized by the use of the present system.

The drain/tube (2), canister (6), disposable pump head (4), purge tube (9), valve (11) optionally filter (12) and filter (7) may be in the form of separate units or some or all of them may be integrated units.

The filter (7) can be a filter of any kind, a membrane or a combination of a filter and a membrane. The filter may be hydrophobic or lipophobic or able to retain bacteria.

Several parameters can be determinative for opening of the valve (11) e.g. time, pressure, power consumption and others.

FIG. 2 shows another embodiment of the invention. The wound is sealed by a wound cover (21) and air and exudate are transported through a first tube (22) by negative pressure created by the pump head (26) and drive unit (28). The first tube (22) can comprise of several parts and different fittings can be added.

The exudate from the first tube (22) is collected in a canister (24). The first tube (22) is connected to the canister (24) through an inlet (23).

In the canister (24) gas and liquid are separated into two phases. The canister (24) can contain an absorbent component. The canister can be any kind of volume suitable for collecting/separate air and liquid.

The gas in the canister (24) is transported through an outlet (25) into the disposable pump head (26) by negative pressure.

A filter (27) can be placed after the disposable pump head (26), between the canister (24) and the disposable pump head (26) or both before and after the disposable pump head (26).

The disposable pump head (26) is connected to the drive unit (28).

A second tube (29) is connected to the sealed wound or semi sealed wound (21) in one end and by an inlet (30) to a first valve (31) and filter (32) in the other end.

Now and then the first valve (31) will open and air will pass through the filter (32), second tube (29), first tube (22) and further through the rest of the system providing a first tube (22) without any liquid strings.

The first tube (22), canister 24), disposable pump head (26), second tube (29), first valve (31), filter (32) and filter (27) can be separate unit or some or all of them can be integrated units.

FIG. 3 discloses a detailed view of an embodiment including two valves. The first tube (2) is connecting the enclosure (34) to the pump (not shown), and the second tube (9) is connecting the first valve (11) to the first tube near the enclosure (34). A second valve (33) is provided at the first tube, between the connection to the second tube (9) and the enclosure (34).

The second valve (33) may be open to fluids moving from the wound enclosure (34) towards the pump but not the other direction. When the first (11) valve is opened, the negative pressure decreases in the tubes (9, 2) and the valve (33) will close. Thus, the negative pressure over the wound will be maintained substantially unaffected by the opening of the first valve. When the first valve (11) is closed again, the negative pressure in the tubes (2, 11) increases, due to the work of the pump, and the second valve will open again.

FIG. 4 discloses a system for vacuum treatment of a wound. The system comprises a wound cover 100 sealed to the tissue around a wound 102, a suction head 104 that is at least partly placed under the wound cover 100 to drain fluid from the wound. The suction head 104 is connected to a conduit part 106 connecting the wound with a suction device 108 so that when a negative pressure is created by the suction device fluid from the wound is transported via the conduit part 106 up to the suction device 108 e.g. via a tube. The suction device comprises a canister and a drive unit and a pump head. The pump head may e.g. be part of the canister or it may be part of the drive unit. The suction head may be integrated in the conduit part 106. In one embodiment of the invention the conduit part 106 is a tube. Alternatively, the conduit part may comprise a first conduit used for suction of fluid from the wound and a second conduit that e.g. may be used for regulating the pressure in the wound or sensing the pressure in the wound or a combination of both.

A system like the one illustrated in FIG. 4 preferably operates with a flow from the wound around 0.5-1.2 l/min. Other flows may be used.

The canisters shown in FIGS. 5-11 should be conceived as a container for collecting fluids from the wound. Thus even though the pump head, the filters, and the regulator are depicted as separated units, they may be integrated with e.g. the canister or the drive unit. The figures should be conceived as a functional description of a system as laid out in FIG. 4.

FIG. 5 shows the airflow through an embodiment of a system as illustrated in FIG. 4. In this system fluid is sucked from the wound 102 via a first conduit 110 (i.e. 106 in FIG. 4) through a pump inlet into the pump or pump head 112 and out through the pump outlet into the canister 114. Part of the fluid in the canister may exit from the canister to the surroundings via an outlet to the surroundings e.g. passing one or more filters as illustrated in FIG. 5, such as a hydrophobic filter 116 and a charcoal filter 118. The filters may have different function such as reducing odour such as a charcoal filter, and an antibacterial filter such as a hydrophobic filter. If the pressure in the wound 102 during use decreases to a predetermined pressure level, preferably the desired treatment pressure, the regulator 120 allows air from the surroundings and or gas originating from the canister 114 to enter into the wound.

FIGS. 6-9 illustrate various conditions for a system like the one illustrated in FIG. 5.

FIG. 6 illustrates evacuation and standard operation of a system according to the one shown in FIG. 5. During evacuation the regulator 120 does not allow air from the surroundings or the canister 114 to enter into the wound 102. Evacuation stops when a predetermine pressure is reached, the predetermined pressure may preferably be the wanted treatment pressure in the wound, though the predetermined pressure may refer to a pressure at another location in the system, so that it indirectly reflects the pressure in the wound. The predetermined pressure should always reflect the wanted treatment pressure in the wound, i.e. the predetermined pressure is based on where in the system the regulator get its pressure input, so that the predetermined pressure is calibrated in order to reflect the wanted treatment pressure.

After evacuation the system enters to standard operation, when the pressure input to the regulator 120 reaches the predetermined pressure. During standard operation gas may start circulating in the system, so that no gas is emitted to the surroundings. During standard operation only small variations in the pressure in the wound appears since the regulator 120 ensures that the gas from the canister 114 is led into the wound 102, if the input pressure to the regulator 120, and thus also the pressure in the wound, falls below the predetermined pressure. Likewise the regulator 120 ensures that no gas is led into the wound if the input pressure to the regulator exceeds the predetermined pressure. Examples of small variations in the pressure in the wound may be around 15 mm Hg when a treatment pressure of 125 mm Hg in the wound is chosen.

In FIG. 6, the bold lines indicate standard operation flow paths, whereas the dashed lines indicate evacuation flow paths.

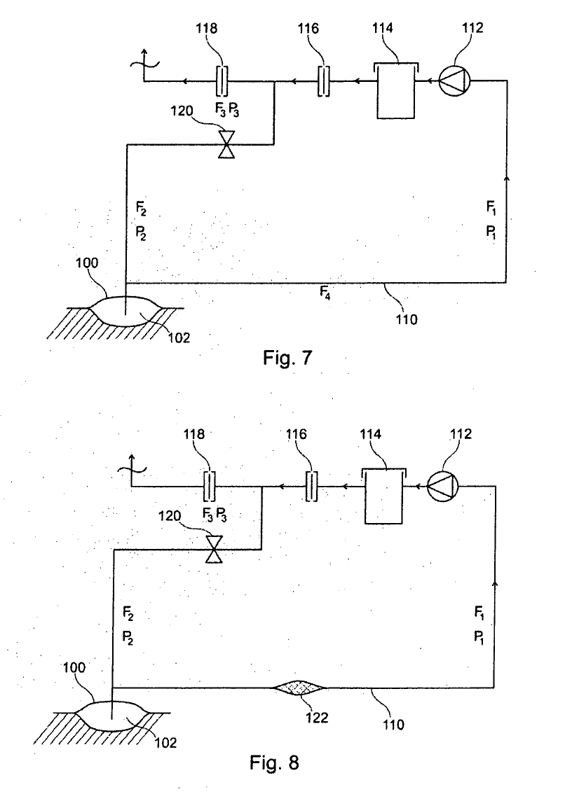

FIG. 7 illustrates how the system operates if a leak e.g. in the wound cover appears. If the system operates with a flow rate F₁ of about 1.2 l/min the leakage flow F₄ may be up to about 0.9 l/min before it is not possible for the system to keep the predetermined pressure as the input pressure. When the system is still functioning despite of the leak, the leakage flow F₄ will be same as the flow to the surroundings F₃. As long as the input pressure to the regulator does not exceed above the predetermined pressure the system will function, so that gas is let into the wound via the regulator flow F₂ if the input pressure to the regulator decreases the predetermined pressure. The system will thus function as long as flow rate F₁ equals the sum of regulator flow F₂ and the flow to the surroundings F₃.

FIG. 8 illustrates what happens if a conduit part 110 connecting the wound and the pump head 112 has an occlusion 122 caused by the fluid from the wound 102. In an embodiment of the invention the drive unit stops operating the pump head 112 when the input pressure is around the predetermined pressure, the flow rate F₁ and the regulator flow F₂ equals 0 since this means that the occlusion 122 is stuck in the conduit part 110. In this case the pump head pressure p₁ will be lower than the pressure in the wound and thus a force will be applied to the occlusion 112, which may cause the occlusion to move towards the pump head 112 even when the pump head is not operating, and in this way it may be possible to remove the occlusion in the conduit 110. Alternatively if the pressure at the pump inlet and in the conduit part p₁ between the occlusion and the pump inlet decreases to a preset occlusion pressure, such as e.g. 300 mm Hg, the drive unit will stop operating the pump head 112. In an embodiment of the invention the drive unit will start operating the pump head after a predetermined period of time. In another embodiment the regulator 120 detects if the input pressure falls below the predetermined pressure after the drive unit has stopped operating the pump 112, and the regulator ensures that the drive unit starts operating the pump head 112.

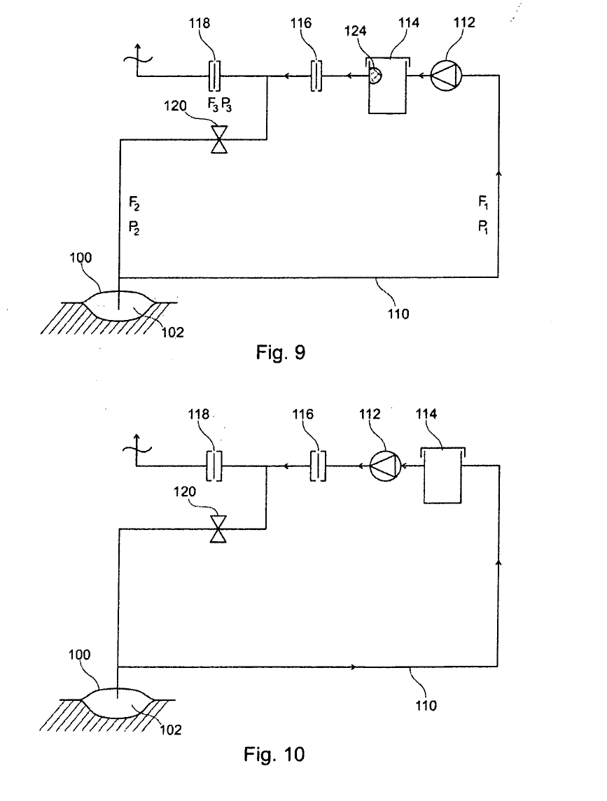

FIG. 9 illustrates occlusion of a part of the path to the outlet to the surroundings. Occlusion may for example occur in a case where a filter is placed before the outlet to the surroundings in the canister 114 and the filter becomes occluded. In FIG. 9, the occlusion is illustrated at 124. A stop criterion for such a situation could be when the flow rate F₁ and the regulator flow F₂ equals 0 and the pump head pressure p₁ and input pressure p₂ equals the pressure of the surroundings.

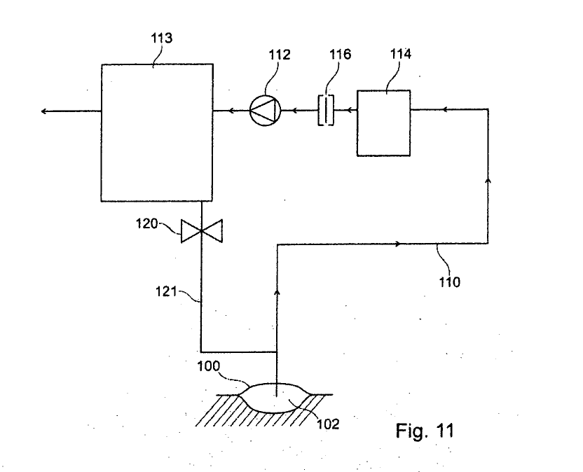

FIG. 10 shows the airflow through another embodiment of a system as illustrated in FIG. 4. In this embodiment, compared to the one shown in FIG. 5, the pump head 112 is placed after the canister 114 and before an antibacterial filter 116.

FIG. 11 shows the airflow through an embodiment of a system as illustrated in FIG. 4. In this embodiment, the pump head 112 placed after the canister 114 and after at least one filter 116 and/or 118. The filter may include at least one of an odour filter and an antibacterial filter. The pump head is driven by a drive unit 113. Pressure sensor 121 is provided between the regulator 120 and the wound 102 to measure a pressure at or near the wound.

FIG. 12 shows a canister 114 that e.g. may be used in a system as illustrated in FIG. 5. In this embodiment of a canister the pump head is part of the canister, the pump head having a pump inlet 126 and a pump outlet 128. The canister also has a wound inlet 130, which in use is fluidically connected with wound. The wound inlet 130 and the pump inlet 126 are connected via an integral canister conduit. The canister further has an outlet 132 to the surroundings and a regulator outlet 120, which in use is fluidically connected to the wound. Reference numbers 1-16 in FIG. 12 refer to the following features:

Canister Shell, which may be either be may of a hard material,
1 such as a plastic or a soft bag e.g. made of a plastic
2 Antibacterial filter such as a charcoal filter
3 Filter Housing
4 Odour Filter such as a Hydrophobic Filter
5 Filter Cover
6 Silencer in form of a silencer Disc
7 One-Way Valve
8 One-Way Valve
9 Pump head retainer such as a Diaphragm Retainer
10 Canister Diaphragm
11 Canister Pump Plate
12 Regulator Plate Seal
13 Regulator Pressure Plate
14 Regulator Spring
15 Set Screw
16 Gel pack

In an embodiment of the canister the force of the pressure spring cannot be changed thus securing a fixed treatment pressure. In another embodiment of the canister the force of the pressure spring may be varied resulting in different treatment pressures, so that when a certain treatment pressure has been chosen the force of pressure spring cannot be changed e.g. by use of a regulator element in form of a lock, hereby securing a fixed treatment pressure.

1. A canister for collection of effluents, in particular exudates, from a wound during vacuum treatment, said canister comprising
an outlet to the surroundings,
a wound inlet,
a pump head having a pump inlet and a pump outlet, and
a regulator regulating the pressure in the wound, the regulator securing a fixed treatment pressure.

2. A canister according to claim 1, wherein the regulator is non-detachably integrated within a housing of the canister.

3. A canister according to claim 1, the regulator further comprising a regulator element and wherein the fixed treatment pressure can be chosen among a number of treatment pressures in the regulator and wherein the chosen treatment pressure is secured by the regulator element thereby securing the fixed treatment pressure to be the chosen treatment pressure.

4. A canister according to claim 1, wherein the regulator regulates the pressure by sensing the pressure in the wound and in response hereto regulates the operation speed of suction of the pump head.

5. A canister according to claim 1, wherein the regulator is arranged to allow air into the wound if the pressure at the wound is less than the fixed treatment pressure, so as to increase the pressure in the wound.

6. A canister according to claim 5, wherein the regulator further comprises a regulator spring and a regulator outlet, such that if in use the pressure at the wound is less than the fixed treatment pressure, air is allowed through the regulator outlet and into the wound.

7. A canister according to claim 5, wherein the regulator is arranged to draw gas from the canister into the wound.

8. A canister according to claim 5, wherein the regulator is arranged to draw ambient air into the wound.

9. A canister according to claim 5, wherein the regulator further comprises a filter element thereby securing that only non-contaminated gas is let into the wound.

10. A canister according to claim 1, wherein the wound inlet and the pump inlet are connected such that the pressure in the canister is approximately the same as the pressure in the surroundings.

11. A canister according to claim 1, wherein the wound inlet is connected to a part of the shell of the canister such that in use the pressure inside the canister is less than the pressure at the surroundings, and that the pump inlet is in contact with the inside of canister, such that in use canister fluid is sucked into the pump head via the pump inlet and wound fluid is sucked from the wound out through the wound inlet to the inside of the canister thus mixing the wound fluid into the canister fluid.

12. A suction device for removal of effluents, in particular exudates, from a wound during vacuum treatment, the suction device comprising
a canister with a wound inlet,
a pump head having a pump inlet and a pump outlet
a drive unit operating the pump head,
an outlet to the surroundings, and
a regulator regulating the pressure at the wound, the regulator securing a fixed treatment pressure.

13. A suction device according to claim 12, the regulator further comprising a regulator element and wherein the fixed treatment pressure can be chosen among a number of treatment pressures in the regulator and that the chosen treatment pressure is secured by the regulator element thereby securing the fixed treatment pressure to be the chosen treatment pressure.

14. A suction device according to claim 12, wherein the regulator regulates the pressure by sensing the pressure in the wound and in response hereto regulates the operation speed of suction of the pump head.

15. A suction device according to claim 12, wherein the regulator is arranged to allow air into the wound if the pressure at the wound is less than the fixed treatment pressure, so as to increase the pressure in the wound.

16. A suction device according to claim 15, wherein the regulator further comprises a regulator spring and a regulator outlet, such that if in use the pressure in the wound is less than the fixed treatment pressure, air is allowed through the regulator outlet and into the wound.

17. A suction device according to claim 15, wherein the regulator is arranged to draw gas from the canister into the wound.

18. A suction device according to claim 15, wherein the regulator is arranged to draw ambient air into the wound.

19. A suction device according to claim 15, wherein the regulator comprises a filter element thereby securing that non-contaminated gas is let into the wound.

20. A suction device for removal of effluents, in particular exudates, from a wound during vacuum treatment, the suction device comprising a canister with a wound inlet, a pump head having a pump inlet and a pump outlet a drive unit operating the pump head an outlet to the surroundings, a regulator regulating the pressure at the wound, the regulator securing a fixed treatment pressure, and wherein the canister is one according to claim 1.

21. A system for vacuum treatment of a wound, the system comprising
a suction device according to claim 12,
a wound cover, and
a conduit part connecting the wound cover and the wound inlet.

22. A system according to claim 21, wherein the conduit part comprises a first conduit and a second conduit.

23. A system according to claim 22, wherein the first conduit is used for suction of fluid from the wound.

24. A system according to claim 22, wherein the second conduit is used for regulating the pressure in the wound.

25. A system according to claim 21, further comprising a suction head at least partly placed under the wound cover.

26. A system according to claim 25, wherein the suction head is connected to the conduit part.

27. A system according to claim 21, wherein the conduit part comprises a suction head at least partly placed under the wound cover, a first conduit and a second conduit, the first conduit being connected with the suction head.
